(19) United States
(12) Plant Patent Application Publication
(10) Pub. No.: US 2002/0188987 P1
(43) Pub. Date: Dec. 12, 2002

(54) Gladiolus plant named '5-11'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/877,572

(22) Filed: Jun. 8, 2001

(51) Int. Cl. A01H 5/00

(52) U.S. Cl. PLT/301

(57) ABSTRACT

A new and distinct gladiolus variety, designated ‘5-11’, is shown and described. Compared to the ‘T-200’ variety, the ‘5-11’ variety is 25 cm taller and will maintain one to two more flowers in open bloom. The ‘5-11’ variety is also able to maintain up to six to seven flowers in open bloom simultaneously, beginning with a tight cut stem.

The present invention comprises a new and distinct variety of a Gladiolus plant referred to by the variety name ‘5-11.’

### BRIEF DESCRITION OF THE PHOTOGRAPHS

FIGURE 1 is a photograph of a ‘5-11’ variety plant in bloom.

### ASEXUAL REPRODUCTION

The new variety was originated by the Applicant in a controlled proprietary breeding program in Ft. Myers, Fla. wherein selected gladiolus varieties were crossed. The female parent was a gladiolus variety named ‘Dr. Magee,’ characterized in part by having a small pink bloom, a short stem having a short flower head, and high resistance to Fusarium fungi species. The male parent was a white gladiolus variety named ‘T-200,’ an unpatented, released variety by Turk characterized in part by having a harvesting time similar to the ‘White Friendship’ gladiolus variety, 16 to 18 florets, and lightweight stems. The seeds were planted in Ft. Myers, Fla., and the selection of the ‘5-11’ variety was made in Spring 1988. Asexual reproduction of the ‘5-11’ variety was achieved by collecting cormels from the first corm. All subsequent asexual reproductions of the ‘5-11’ variety remain true to the original variety type.

### BOTANICAL DESCRIPTION

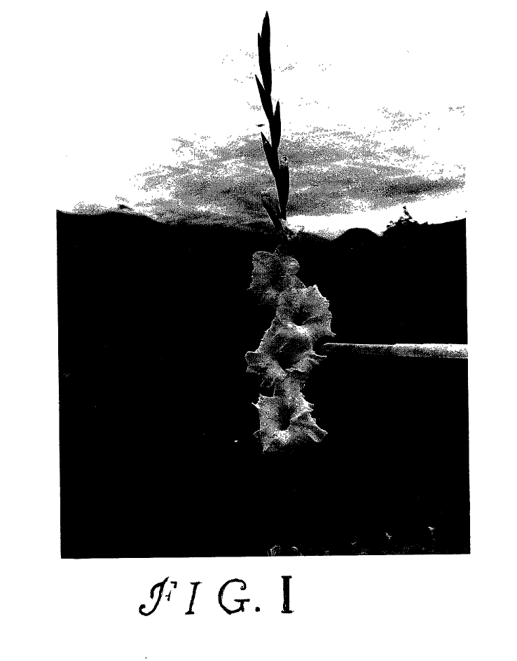

The accompanying color photograph (FIGURE 1) shows the inflorescence and various stages of blooming of the ‘5-11’ variety plant.

The bud size of the ‘5-11’ variety is about 6 cm in length. The color of the corolla bloom is a pure white. The diameter of the entire bloom is about 13 cm.

The spike of the plant is about 122 cm and comprises 17 florets when grown from #2 size corms (1.25-1.50 inches) in Ecuador. The head length is about 49 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes).

The corms of the ‘5-11’ variety are typical for the gladiolus The leaf color of the ‘5-11’ variety is green. The leaf size is about 74 cm (length) by 3 cm (width).

### GENERAL OBSERVATIONS

The ‘5-11’ variety takes about 90 days to harvest in Ecuador, which is similar to the gladiolus variety ‘Advance.’ The ‘5-11’ variety grows all year; however, growth is best during longer day lengths (i.e. 12-16 hours). The ‘5-11’ variety tolerates high temperatures well without burning.

The foliage is upright standing and usually produces one to two stems per Jumbo size corm. The variety can maintain up to six to seven flowers in open bloom simultaneously in a vase of water and can be harvested with one flower showing color. The variety is an excellent opener after transport in that the stems may be cut tight, shipped dry for a week, and still bloom well afterwards. The variety is also a slow opener.

The ‘5-11’ variety has good resistance to attack by Fusarium fungi and foliar diseases.

Compared to the T-200 parent variety, the ‘5-11’ variety is 25 cm taller, has a larger bloom size that is about 2 cm wider in diameter, can hold one to two more blooms open, and is about two weeks slower to harvest. Disease resistance is similar to that of the ‘T-200’ variety.

I claim:

1. A new and distinct gladiolus variety ‘5-11’, as shown and described herein.
